(19) United States
(12) Patent Application Publication
(10) Pub. No.: US 2020/0140446 A1
(43) Pub. Date: May 7, 2020

(54) 5,8-DIMETHYL-2-[2-(1-METHYL-4-PHENYL-1H-IMIDAZOL-2-YL)-ETHYL]-[1,2,4]TRIAZOLO[1,5-A]PYRAZINE HEMIADIPATE

(71) Applicant: H. Lundbeck A/S, Valby (DK)

(72) Inventors: Anette Frost Jensen, Frederiksberg C. (DK); Lars Ole Lyngsø, Veksø Sj. (DK); Flemming Elmelund Nielsen, Virum (DK)

(73) Assignee: H. Lundbeck A/S, Valby (DK)

(21) Appl. No.: 16/674,717

(22) Filed: Nov. 5, 2019

(30) Foreign Application Priority Data
Nov. 6, 2018 (DK) PA201800825
Aug. 2, 2019 (EP) 19189761.0

(51) Int. Cl. C07D 487/04; A61K 9/16; A61P 25/18

(52) U.S. Cl. CPC C07D 487/04; C07B 2200/13; A61P 25/18; A61K 9/1682

(57) ABSTRACT

The present invention relates to 5,8-Dimethyl-2-[2-(1-methyl-4-phenyl-1H-imidazol-2-yl)-ethyl]-[1,2,4]triazolo[1,5-a]pyrazine hemiadipate, which is non-hygroscopic. The present invention also relates to pharmaceutical compositions comprising 5,8-Dimethyl-2-[2-(1-methyl-4-phenyl-1H-imidazol-2-yl)-ethyl]-[1,2,4]triazolo[1,5-a]pyrazine hemiadipate, as well as the use of 5,8-Dimethyl-2-[2-(1-methyl-4-phenyl-1H-imidazol-2-yl)-ethyl]-[1,2,4]triazolo[1,5-a]pyrazine hemiadipate in therapy. The 5,8-Dimethyl-2-[2-(1-methyl-4-phenyl-1H-imidazol-2-yl)-ethyl]-[1,2,4]triazolo[1,5-a]pyrazine hemiadipate being depicted below.

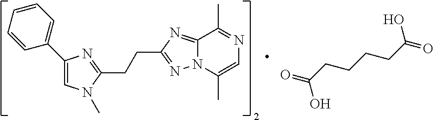

### FIELD OF THE INVENTION

The present invention relates to 5,8-Dimethyl-2-[2-(1-methyl-4-phenyl-1H-imidazol-2-yl)-ethyl]-[1,2,4]triazolo[1,5-a]pyrazine hemiadipate, which is a novel non-hygroscopic solid form of 5,8-Dimethyl-2-[2-(1-methyl-4-phenyl-1H-imidazol-2-yl)-ethyl]-[1,2,4]triazolo[1,5-a]pyrazine. The present invention also relates to pharmaceutical compositions comprising 5,8-Dimethyl-2-[2-(1-methyl-4-phenyl-1H-imidazol-2-yl)-ethyl]-[1,2,4]triazolo[1,5-a]pyrazine hemiadipate, as well as the use of 5,8-Dimethyl-2-[2-(1-methyl-4-phenyl-1H-imidazol-2-yl)-ethyl]-[1,2,4]triazolo[1,5-a]pyrazine hemiadipate in therapy.

### BACKGROUND OF THE INVENTION

The compound 5,8-Dimethyl-2-[2-(1-methyl-4-phenyl-1H-imidazol-2-yl)-ethyl]-[1,2,4]triazolo[1,5-a]pyrazine is a potent Phosphodiesterase 10A (PDE10A) inhibitor, and the free base of this compound was first described in WO2009/152825 (Example 12). 5,8-Dimethyl-2-[2-(1-methyl-4-phenyl-1H-imidazol-2-yl)-ethyl]-[1,2,4]triazolo[1,5-a]pyrazines chemical structure is presented below and denoted Compound (I). WO2009/152825 also describes a method for obtaining the free base of Compound (I).

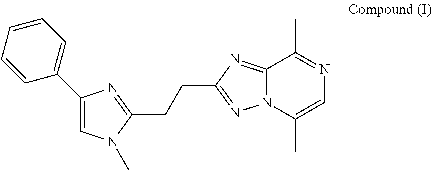 [SMILES: Cc1ncc(C)n2nc(CCc3nc(-c4ccccc4)cn3C)nc12]

PDE10A is an enzyme that hydrolyses the signalling molecules cAMP and cGMP to their inactive forms; 5′AMP and 5′GMP, respectively (Fujishige K. et al., 1999, J Biol Chem., 274(26): 18438-18445), and PDE10A inhibitors have been proposed to represent a new mechanism of action useful for treatment of all symptom domains in schizophrenia; positive symptoms, negative symptoms and cognitive dysfunction (Kehler and Nielsen, 2011, Current Pharmaceutical Design, vol. 17:137-150).

### SUMMARY OF THE INVENTION

During pharmaceutical development, it was found that the free base of Compound (I) was very hygroscopic and that at least two hydrate forms exists. For these reasons the free base of Compound (I) was deemed unsuitable for further development and consequently this left an unmet need for a solid form of Compound (I), suitable to be advanced into clinical development.

The present invention relates to a novel solid non-hygroscopic form of 5,8-Dimethyl-2-[2-(1-methyl-4-phenyl-1H-imidazol-2-yl)-ethyl]-[1,2,4]triazolo[1,5-a]pyrazine also referred to as Compound (I) throughout this application. Specifically, the present invention relates to the Compound (I) hemiadipate per se and to pharmaceutical compositions comprising said Compound (I) hemiadipate for use in therapy.

The Compound (I) hemiadipate is represented by the molecular structure below and is composed of 2 equivalents of Compound (I) and 1 equivalent of adipic acid.

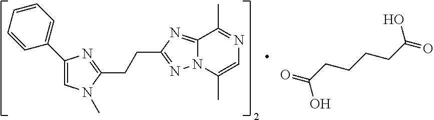

In one embodiment, the invention relates to said Compound (I) hemiadipate for use in the treatment of persistent negative symptoms or persistent prominent negative symptoms, in particular in schizophrenia.

In one embodiment, the invention relates to said Compound (I) hemiadipate for use in the treatment of cognitive dysfunctions, in particular in schizophrenia.

In one embodiment, the invention relates to a method of the treatment of negative symptoms, persistent negative symptoms, prominent negative symptoms, persistent prominent negative symptoms, predominant negative symptoms, persistent predominant negative symptoms and/or cognitive dysfunctions in particular in schizophrenia, which method comprises administration of a therapeutically effective amount of said Compound (I) hemiadipate.

In one embodiment, the invention relates to the use of said Compound (I) hemiadipate in the manufacture of a medicament.

In one embodiment, the invention relates to the use of said Compound (I) hemiadipate in the manufacture of a medicament for use in the treatment of negative symptoms, persistent negative symptoms, prominent negative symptoms, persistent prominent negative symptoms, predominant negative symptoms, persistent predominant negative symptoms and/or cognitive dysfunctions.

In one embodiment, the invention relates to a solid oral dosage form comprising said Compound (I) hemiadipate.

### Definitions

In the present context, expressions like “Compound (I) hemiadipate characterized by the XRPD shown in FIG. 1A” is meant to describe the crystalline form of Compound (I) hemiadipate which is identifiable with reference to a XRPD substantially similar to FIG. 1A, i.e. exhibiting an XRPD pattern with reflections substantially at the angles as exemplified in FIG. 1A when measured under comparable conditions.

The term “and/or” as used in statements like “persistent negative symptoms and/or persistent prominent negative symptoms and/or cognitive dysfunctions” are meant to indicate a pathological state, wherein the patient has either persistent negative symptoms, persistent prominent negative symptoms or cognitive dysfunctions, or a combination of two or all three of these types of symptoms, which are further defined hereinbelow.

The term “negative symptoms” as used herein is meant to describe one or more symptom(s) associated with a CNS disorders, in particular schizophrenia. Specific examples of such symptoms are: alogia, amotivation, anhedonia, asociality, emotional withdrawal, social withdrawal, difficulty in abstract thinking, lack of spontaneity and flow of conversation, stereotyped thinking, affective flattening, avolition, poverty of speech, dysphoric mood, lack of insight and demoralisation.

The term “persistent negative symptoms” is meant to describe a clinical state of a patient where the negative symptoms have been persistent for at least 6 months.

The term “prominent negative symptoms” is meant to describe a clinical state of a patient where the negative symptoms are a prominent part of the patient's clinical presentation.

The term “persistent prominent negative symptoms” are meant to describe “prominent negative symptoms”, as defined above, that have been persistent for at least 6 months.

The patients with “predominant negative symptoms” are meant to describe a subpopulation of patients with “prominent negative symptoms” who display negative symptoms that are at least moderate in severity, as evaluated on an accepted and validated rating scale (e.g. the PANSS negative symptom score, PANSS Marder Negative Symptom Factor score or the Brief Negative Symptom Scale (BNSS)) and display no or only little positive symptoms (e.g. assessed by the PANSS positive symptom score).

The term “persistent predominant negative symptoms” are meant to describe “predominant negative symptoms”, as defined above, that have been persistent for at least 6 months.

The terms “cognitive dysfunction” and “cognitive impairment” are used interchangeably and is meant to describe symptoms affecting cognition such as impaired executive functioning, impaired working memory, attention deficits, social cognitive impairment, jumping to conclusions, Theory of Mind (TOM) impairments, deficits in emotion recognition or affect discrimination.

The term “clinically stable positive symptoms” or “clinically stable phase” as used herein is meant to describe a clinical state of a patient suffering from schizophrenia, who has had no exacerbation of their positive symptoms, within a period of 6 months preceding the onset of treatment with compound (I) hemiadipate. Any positive symptoms, which may be present during the clinically stable phase, should not exceed moderate severity, as evaluated on an accepted and valid rating scale, such as PANSS.

The term “monotherapy” in the present context is meant to describe the treatment regimen related directly to the schizophrenic diagnosis. Hence, when the present invention describes the use of compound (I) hemiadipate as monotherapy, it means that said compound should not be administered as adjunctive/add-on treatment to any background antipsychotic treatment regimens, which may be comprised of either typical or atypical antipsychotics (examples of such drugs are given below).

Consequently, a patient being administered compound (I) hemiadipate as monotherapy for treatment of persistent negative symptoms, prominent negative symptoms, persistent prominent negative symptoms, predominant negative symptom, persistent predominant negative symptoms and/or cognitive impairments may in parallel with such treatment receive other drugs to treat potential additional pathological conditions, which are unrelated to the schizophrenic diagnosis.

In the present invention, “antipsychotic drugs” are meant to describe a specific class of drugs, which is primarily used to manage psychosis. In the present invention, “antipsychotic drugs” may for example be selected from the group comprising haloperidol, pimozide, chlorpromazine, fluphenazine, perazine, perphenazine, trifluoperazine, clopenthixol, thiothixene, loxapine, sultopride, iloperidone, lurasidone, paliperidone, risperidone, ziprasidone, aripiprazole, brexpiprazole, cariprazine, asenapine, clozapine, olanzapine, quetiapine, zotepine, blonanserin, pimavanserin and sertindole.

### BRIEF DESCRIPTION OF DRAWINGS

FIGS. 1A and 1B: XRPD pattern of Compound (I) hemiadipate and the free base of Compound (I). The XRPD diffractogram was obtained using CuKα1 radiation. The y-axis shows the intensity (counts) and the x-axis shows the 2θ-angles (°). A: the XRPD of the only identified form of Compound (I), a crystalline non-hygroscopic, non-hydrated and non-solvated form. B: the two known hydrate forms of the free base of Compound (I), Hydrate I (top) and Hydrate II (bottom).

FIG. 2: DSC thermogram of Compound (I) hemiadipate. The X axis shows the temperature (° C.), the y-axis shows the DSC heat flow in mW.

FIG. 3: DVS diagram of Compound (I) hemiadipate (A) and the free base of Compound (I) (B). The DVS experiment was performed at 25° C. The first y-axis (left) shows the change in mass (%) and the other y-axis (right) shows the targeted relative humidity (RH)(%), while the x-axis show the time in minutes. The thin line shows the changes in target RH and the bold line the changes in relation to mass.

FIG. 4: Effect of Compound (I) in the Conditioned Avoidance Response (CAR) assay.

### DETAILED DESCRIPTION OF THE INVENTION

The present invention provides the non-hygroscopic Compound (I) hemiadipate represented by the chemical structure below. The compound (I) hemiadipate is composed of 2 equivalents of Compound (I) and 1 equivalent of adipic acid.

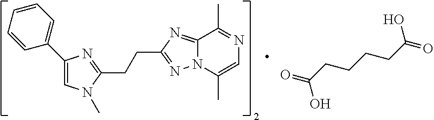

The inventors have found that the free base of Compound (I) is unfit for pharmaceutical development because this solid form is very hygroscopic and at least two hydrates exists. The inventors of the present invention have unexpectedly identified Compound (I) hemiadipate as being a non-hygroscopic form of Compound (I). In addition, the Compound (I) hemiadipate adopts only one known polymorphic form and does not form neither hydrates nor solvates. These properties establish that the Compound (I) hemiadipate is superior to known solid forms of Compound (I) and particularly well suited as an active pharmaceutical ingredient (API). Furthermore, the Compound (I) hemiadipate is also found to have a more optimal dissolution profile compared to the free base of Compound (I).

Within the pharmaceutical industry, it is well known that hygroscopic solids possess the risk of absorbing moisture on storage and must therefore be packed in containers free of moisture or stored under very dry conditions, which is quite costly. Furthermore, hygroscopic solids are more difficult to handle during processing into for example pharmaceutical products due the risk of absorbing moisture which may influence the quality of the end-product, especially if this is a solid oral dosage form. These disadvantages are avoided by using, the non-hygroscopic Compound (I) hemiadipate. The non-hygroscopic property increases the stability of the solid form and may facilitate longer shelf-life and stability of the final dosage form, while at the same time reducing costs associated with formulation and packaging as well as storage conditions. The non-hygroscopic characteristic also makes the Compound (I) hemiadipate particularly well suited for multiple methods known in API processing, such as wet granulation and direct compression. Compound (I) hemiadipate is characterized by being non-hygroscopic, meaning that this solid form does not absorb water when exposed to a relative humidity (RH) up to about 95% at about 25° C.

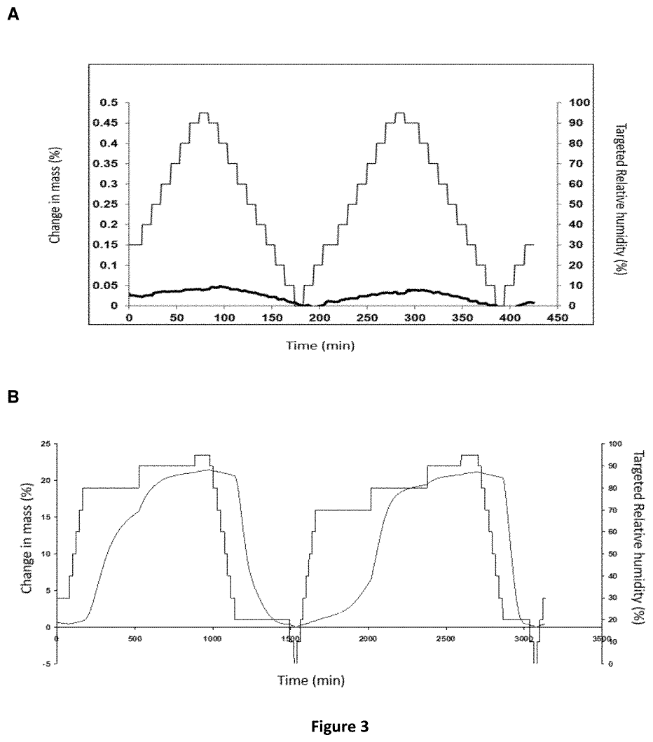

As shown in Example 4 of the present invention, DVS (Dynamic Vapour Sorption) experiments were performed at about 25° C. The results showed that substantially no water was absorbed by the Compound (I) hemiadipate even at about 95% RH, on the contrary, the free base of Compound (I) is clearly very hygroscopic (FIG. 3).

It is therefore an important characteristic of the Compound (I) hemiadipate that it is non-hygroscopic, as defined above. Thus, the present invention relates to Compound (I) hemiadipate which is non-hygroscopic, meaning that it absorbs less than about 1.5% of its weight in moisture when exposed to about 95% RH at about 25° C., such as less than about 1.0%, such as less than about 0.5%, such as less than about 0.3% moisture is absorbed. In one embodiment, the Compound (I) hemiadipate absorbs less than about 0.1% of its weight in moisture when exposed to about 95% RH at about 25° C.

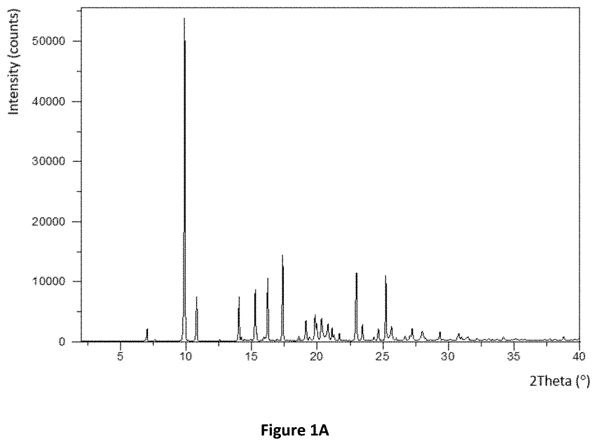

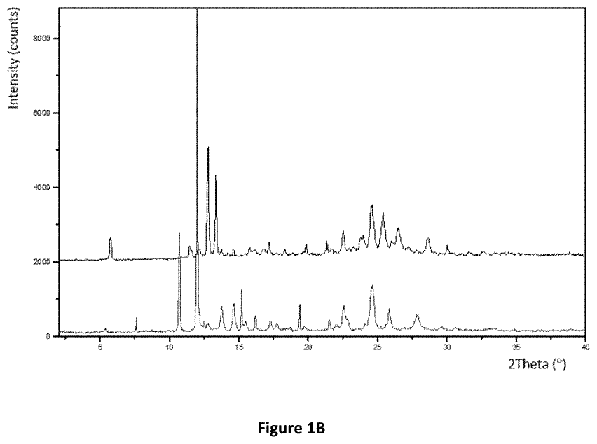

Moreover, besides being non-hygroscopic the Compound (I) hemiadipate appears to adopt only one single crystalline form. This is also highly advantages in many aspects of pharmaceutical processing because it facilitates more control and predictability of the end-product. The Compound (I) hemiadipate has been characterized by X-ray powder diffractogram (XRPD) according to example 2 and the resulting XRPD diffractogram is depicted in FIG. 1; characteristic main reflections are at the following 2θ-angles 7.0, 9.9, 10.8, 14.0, 15.3, 16.2, 17.4, 19.1, 19.8, 20.3 (±0.1° 2θ).

The Compound (I) hemiadipate has also been found not to form hydrates when precipitated from pure water at room temperature, which is further described in example 5. The non-hydrate forming Compound (I) hemiadipate of the present invention has the advantages that it is easy to work with, both from the perspective of chemical production and pharmaceutical production and storage. For example, certain pharmaceutical processes such as granulation by high shear mixing or fluid bed processing implies that the Compound (I) hemiadipate will be partly or fully dissolved in the granulation liquid. This would, if the Compound (I) hemiadipate was hydrate-forming induce a risk of converting the solid form into a hydrated solid form. The subsequent drying of the granulate holds the risk that a hydrated solid form would lose water and be converted into a less hydrated solid form. Consequently, such changes might alter the stoichiometry during processing which has significant drawbacks, such as the risk of obtaining an end-product not fulfilling the product specifications.

Many pharmaceutical processes possess the risk of either hydrate formation or loss of water from a hydrate e.g. wet granulation; fluid bed processing; drying at elevated temperature such as at a temperature in the range of 60−90° C.; aqueous based spray drying; aqueous based coating of granules, pellets or tablets; milling at elevated temperature, such as at a temperature in the range of 60−150° C. In this view, employing the non-hydrate forming Compound (I) hemiadipate as the API would provide the pharmaceutical development process with a higher degree of freedom, i.e. leave more options to design the best possible process for the compound.

Also in chemical processing, avoidance of hydrate forming solid forms could be advantageous from a process point of view as it enables the use of water as a solvent in the purification process, and also as a solvent for precipitation without the risk of hydrate formation.

The Compound (I) hemiadipate were also found to be a non-solvate forming solid form of Compound (I), which was assessed in the following solvents: acetone, acetonitrile, methanol, ethanol, 1-propanol, 2-propanol, 1-butanol, 2-butanol, ethyl acetate, isopropyl acetate, methyl isobutyl ketone, tetrahydrofuran, diethylether, N-methylpyrrolidone, dichloromethane, and n-heptane. The non-solvate forming property of the Compound (I) hemiadipate of the present invention, which is further described in example 6, entails advantages in relation to the chemical processing e.g. the lack of solvate formation makes it possible to select the optimal organic solvent for the crystallization process and thereby optimize the purification and yield.

The Compound (I) hemiadipate have several characteristics that makes it superior to the free base in terms of pharmaceutical development, for example the Compound (I) hemiadipate has a dissolution profile, which is favored over the free base dissolution profile, because it provides the opportunity of a more optimal release of the API from the solid dosage form. The Intrinsic dissolution rates of Compound (I) hemiadipate as well as the free base of Compound (I) is further described in example 7.

The many advantages of the Compound (I) hemiadipate is mainly acknowledged in the pharmaceutical formulation process. In these processes, it is highly preferable to work with an API, which is stable and easy to control. In this regard the Compound (I) hemiadipate has unexpectedly been found by the inventors to be the most attractive solid form of Compound (I) for further pharmaceutical development.

The Compound (I) hemiadipate has superior properties over at least 20 other solid forms of compound (I) including the free base. These properties are summarized in table 2.

### Pharmaceutical Compositions and Excipients:

The present invention also provides a process for making a pharmaceutical composition comprising the Compound (I) hemiadipate. The pharmaceutical compositions according to the invention may be formulated with pharmaceutically acceptable excipients in accordance with conventional techniques such as those disclosed in Remington, The Science and Practice of Pharmacy, 22^{th} edition (2013), Edited by Allen, Loyd V., Jr.

Pharmaceutical compositions for oral administration include solid oral dosage forms such as tablets, capsules, powders and granules; and liquid oral dosage forms such as solutions, emulsions, suspensions and syrups as well as powders and granules to be dissolved or suspended in an appropriate liquid.

Pharmaceutical compositions for parenteral administration include sterile aqueous and non-aqueous solutions, dispersions, suspensions or emulsions for injection or infusion, concentrates for injection or infusion as well as sterile powders to be reconstituted in sterile solutions or dispersions for injection or infusion prior to use.

Other types of pharmaceutical compositions include suppositories, inhalants, creams, gels, dermal patches, implants and formulations for buccal or sublingual administration.

### Definition of “Excipient”:

In the present context, “excipient” or “pharmaceutically acceptable excipient” refers to pharmaceutical excipients including, but not limited to, fillers, antiadherents, binders, coatings, colours, disintegrants, flavours, glidants, lubricants, preservatives, sorbents, sweeteners, solvents, vehicles and adjuvants.

Examples of excipients suitable for solid oral formulation include, but are not limited to, microcrystalline cellulose, corn starch, lactose, mannitol, povidone, croscarmellose sodium, sucrose, cyclodextrin, talcum, gelatine, pectin, magnesium stearate, stearic acid and lower alkyl ethers of cellulose. Similarly, the solid formulation may include excipients for delayed or extended release formulations known in the art, such as glyceryl monostearate or hypromellose.

Examples of excipients suitable for liquid oral formulation include, but are not limited to, ethanol, propylene glycol, glycerol, polyethylenglycols, poloxamers, sorbitol, poly-sorbate, mono and di-glycerides, cyclodextrins, coconut oil, palm oil, and water.

Further excipients may be used in solid and liquid oral formulations, such as colourings, flavourings and preservatives etc.

Examples of excipients suitable for parenteral formulation include, but are not limited to water, coconut oil, palm oil and solutions of cyclodextrins. Aqueous formulations should be suitably buffered if necessary and rendered isotonic with sufficient saline or glucose.

It is requisite that the excipients used for any pharmaceutical formulation comply with the intended route of administration and are compatible with the active ingredients.

### Administration Routes and Dosage Forms:

The pharmaceutical compositions may be specifically formulated for administration by any suitable route such as the oral, rectal, nasal, buccal, sublingual, transdermal and parenteral (e.g. subcutaneous, intramuscular, and intravenous) route; the oral route being preferred.

It will be appreciated that the route will depend on the general condition and age of the subject to be treated, the nature of the condition to be treated and the active ingredient.

Solid oral dosage forms may be presented as discrete units (e.g. tablets or hard or soft capsules), each containing a predetermined amount of the active ingredient, and preferably one or more suitable excipients. Where appropriate, the solid dosage forms may be prepared with coatings such as enteric coatings or they may be formulated so as to provide modified release of the active ingredient such as delayed or extended release according to methods well known in the art. Where appropriate, the solid dosage form may be a dosage form disintegrating in the saliva, such as for example an orodispersible tablet.

If solid material is used for oral administration, the formulation may for example be prepared by mixing the active ingredient with solid excipients and subsequently compressing the mixture in a conventional tableting machine; or the formulation may for example be placed in a hard capsule e.g. in powder, pellet or mini tablet form. The amount of solid excipient will vary widely but will typically range from about 25 mg to about 1 g per dosage unit.

Liquid oral dosage forms may be presented as for example elixirs, syrups, oral drops or a liquid filled capsule. Liquid oral dosage forms may also be presented as powders for a solution or suspension in an aqueous or non-aqueous liquid.

Liquid oral dosage forms may for example be prepared by dissolving or suspending the active ingredient in an aqueous or non-aqueous liquid, or by incorporating the active ingredient into an oil-in-water or water-in-oil liquid emulsion.

### Doses:

In one embodiment, the Compound (I) hemiadipate of the present invention is administered in an amount from about 0.001 mg/kg body weight to about 100 mg/kg body weight per day. In particular, daily dosages may be in the range of about 0.01 mg/kg body weight to about 50 mg/kg body weight per day. The exact dosages will depend upon the frequency and mode of administration, the sex, the age the weight, and the general condition of the subject to be treated, the nature and the severity of the condition to be treated, any concomitant diseases to be treated, the desired effect of the treatment and other factors known to those skilled in the art.

A typical oral dosage for adults will be in the range of about 0.01-500 mg/day of a compound of the present invention, such as about 0.1-100 mg/day, such as about 0.5-50 mg/day or about 1-25 mg/day. Conveniently, the compounds of the invention are administered in a unit dosage form containing said compounds in an amount of about 0.01 to about 500 mg, such as about 0.5 mg, about 1 mg, about 2 mg, about 3 mg, about 4 mg, about 5 mg, about 6 mg, about 7 mg, about 8 mg, about 9 mg, about 10 mg, about 50 mg about 100 mg, about 150 mg, about 200 mg or about 250 mg of a compound of the present invention.

In the present invention daily oral dosages of about 1-8 mg are preferred and this dose is preferably administered either once or twice daily.

### Compound (I) Hemiadipate for Use in Treatment

The Compound (I) hemiadipate, which is a potent PDE10 inhibitor, is believed to be useful in the treatment of negative symptoms and cognitive impairments. In one embodiment, the patient to be treated with the Compound (I) hemiadipate is suffering from schizophrenia or a schizophrenia related disorder, such as residual, deficit or simple schizophrenia. In a further embodiment, the patient to be treated suffers from schizophrenia and is considered to be in the clinically stable phase. In one embodiment, said clinically stable phase is characterized by the absence of any recent acute exacerbation incidents, which have required hospitalization or change in antipsychotic medication (with reference to change from one specific drug to another or change from one specific dose to another) within the last 6 months. In one embodiment, the clinically stable phase is characterized by a patient who has been treated for schizophrenia with stable doses of an antipsychotic within the approved dose range and without any dose increase during the last 6 months. In one embodiment, the clinically stable phase is characterized by a patient who has had no psychiatric admissions/hospitalization due to clinical deterioration during the last 6 months, provided that the admissions/hospitalization excludes ambulatory visits to ask for advice from the psychiatry team. In one embodiment, the clinically stable phase is characterized by a patient who has not been involved in any violent episodes, including suicide attempts, for the last 12 months.

In one aspect of the present invention, the Compound (I) hemiadipate is used to treat a schizophrenic patient who is treatment-naïve to antipsychotic drugs, i.e. said patient has not previously been treated with any type of antipsychotic. In another aspect, the Compound (I) hemiadipate is used to treat a schizophrenic patient who was previously treated with an antipsychotic drug but discontinued such treatment, e.g. because the drug did not provide adequate improvement in reference to the negative symptoms and/or because the subject could not tolerate the side effects of the drug. In yet another aspect of the invention, the Compound (I) hemiadipate is used to treat a schizophrenic patient who, up until now, has been treated with an antipsychotic drug but such treatment has not provided adequate improvement in reference to the negative symptoms, and the patient is therefore switched to a Compound (I) hemiadipate treatment regimen.

In one embodiment, the Compound (I) hemiadipate is used as monotherapy to treat persistent negative symptoms and/or persistent prominent negative symptoms and/or cognitive impairments in a patient suffering from schizophrenia, who are clinically stable in reference to the positive symptoms. In another embodiment, the Compound (I) hemiadipate is used as monotherapy to treat persistent negative symptoms and/or persistent prominent negative symptoms and/or cognitive impairments in an individual who is in the prodromal phase of schizophrenia.

In one aspect of the present invention, the negative symptoms to be treated are primary negative symptoms. Primary negative symptoms are etiologically related to the core pathophysiology of schizophrenia whereas secondary negative symptoms are derived from other symptoms of schizophrenia, other disease processes, medications, or environment.

### Embodiments

The following embodiments describes the invention in further detail. The embodiments are numbered consecutively, starting from number 1.
E1. A 5,8-Dimethyl-2-[2-(1-methyl-4-phenyl-1H-imidazol-2-yl)-ethyl]-[1,2,4]triazolo[1,5-a]pyrazine hemiadipate.
E2. The Compound (I) hemiadipate according to embodiment 1, represented by the formula

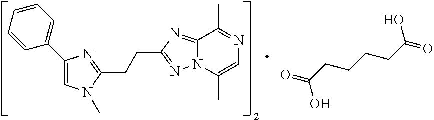

E3. The Compound (I) hemiadipate, according to any of the embodiments 1-2, wherein said Compound (I) hemiadipate comprises 2 equivalents of Compound (I) and about 0.8-1.2 equivalent of adipic acid, such as 2 equivalents of Compound (I) and about 0.9-1.1 equivalent of adipic acid, such as 2 equivalents of Compound (I) and about 0.95-1.05 equivalent of adipic acid, such as 2 equivalents of Compound (I) and about 0.98-1.02 equivalent of adipic acid.
E4. The Compound (I) hemiadipate according to any of embodiments 1-3, wherein said Compound (I) hemiadipate is crystalline.
E5. The Compound (I) hemiadipate according to embodiment 4, which crystal form is characterized by an XRPD obtained using CuKα1 radiation (λ=1.5406 Å) showing peaks at the following 2θ-angles: 7.0, 9.9, 10.8, 14.0, 15.3, 16.2, 17.4, 19.1, 19.8, 20.3 (±0.1° 28).
E6. The Compound (I) hemiadipate according to embodiment 4, which crystal form is characterized by an XRPD obtained using CuKα1 radiation (λ=1.5406 Å) showing peaks at the following 28-angles: 7.0, 9.9, 10.8, 14.0, 15.3, 16.2, 17.4 (±0.1° 28).
E7. The Compound (I) hemiadipate according to any of embodiments 4-6, which crystal form is characterized by an XRPD obtained using CuKα1 radiation (λ=1.5406 Å) as depicted in FIG. 1A.
E8. The Compound (I) hemiadipate according to any of embodiments 4-7, which crystal form is characterized by having a DSC trace showing an endotherm with peak at about 185-187° C.
E9. The Compound (I) hemiadipate according to any of embodiments 1-8, wherein less than about 1.5% moisture is absorbed when said Compound (I) hemiadipate is exposed to about 95% RH at about 25° C.
E10. The Compound (I) hemiadipate according to any of embodiments 1-8, wherein less than about 1.5% moisture is absorbed when said Compound (I) hemiadipate is exposed to about 95% RH at about 25° C., when determined by Dynamic Vapor Sorption (DVS).
E11. The Compound (I) hemiadipate according to any of embodiments 1-8, wherein less than about 1.5% moisture is absorbed when said Compound (I) hemiadipate is exposed to about 95% RH at about 25° C., when determined by Dynamic Vapor Sorption (DVS) according to the method of Example 4.
E12. The Compound (I) hemiadipate according to any of embodiments 1-8, wherein less than about 0.5% moisture is absorbed when said Compound (I) hemiadipate is exposed to about 95% RH at about 25° C.
E13. The Compound (I) hemiadipate according to any of embodiments 1-8, wherein less than about 0.5% moisture is absorbed when said Compound (I) hemiadipate is exposed to about 95% RH at about 25° C., when determined by Dynamic Vapor Sorption (DVS).
E14. The Compound (I) hemiadipate according to any of embodiments 1-8, wherein less than about 0.5% moisture is absorbed when said Compound (I) hemiadipate is exposed to about 95% RH at about 25° C., when determined by Dynamic Vapor Sorption (DVS) according to the method of Example 4.
E15. The Compound (I) hemiadipate according to any of embodiments 1-8, wherein less than about 0.1% moisture is absorbed when said Compound (I) hemiadipate is exposed to about 95% RH at about 25° C.
E16. The Compound (I) hemiadipate according to any of embodiments 1-8, wherein less than about 0.1% moisture is absorbed when said Compound (I) hemiadipate is exposed to about 95% RH at about 25° C., when determined by Dynamic Vapor Sorption (DVS).
E17. The Compound (I) hemiadipate according to any of embodiments 1-8, wherein less than about 0.1% moisture is absorbed when said Compound (I) hemiadipate is exposed to about 95% RH at about 25° C., when determined by Dynamic Vapor Sorption (DVS) according to the method of Example 4.
E18. The Compound (I) hemiadipate according to any of embodiments 1-17 for use as a medicament.
E19. The Compound (I) hemiadipate according to any of embodiments 1-17 for use in therapy.
E20. A pharmaceutical composition comprising the Compound (I) hemiadipate according to any of embodiments 1-17 and at least one pharmaceutically acceptable excipient.
E21. The Compound (I) hemiadipate according to any of embodiments 1-17, for use in treating a patient suffering from persistent negative symptoms associated with schizophrenia, provided that the Compound (I) hemiadipate is administered as monotherapy and wherein the patient has clinically stable positive symptoms.
E22. The Compound (I) hemiadipate according to any of embodiments 1-17, for use in treating a patient suffering from persistent prominent negative symptoms associated with schizophrenia, provided that the Compound (I) hemiadipate is administered as monotherapy and wherein the patient has clinically stable positive symptoms.
E23. The Compound (I) hemiadipate according to any of embodiments 1-17, for use according to embodiments 21-22, wherein the negative symptoms are primary negative symptoms.
E24. The Compound (I) hemiadipate according to any of embodiments 1-17, for use according to any of embodiments 21-22, wherein the negative symptoms are selected from the group comprising: alogia, amotivation, anhedonia, asociality, emotional withdrawal, social withdrawal, difficulty in abstract thinking, lack of spontaneity and flow of conversation, stereotyped thinking, affective flattening, avolition, dysphoric mood, lack of insight, and demoralisation.
E25. The Compound (I) hemiadipate according to any of embodiments 1-17, for use according to embodiments 21-22, wherein the negative symptoms are selected from social withdrawal, anhedonia and/or avolition.
E26. The Compound (I) hemiadipate according to any of embodiments 1-17, for use treating a patient suffering from cognitive impairments associated with schizophrenia, provided that the Compound (I) hemiadipate is administered as monotherapy and wherein the patient has clinically stable positive symptoms.
E27. The Compound (I) hemiadipate according to any of embodiments 1-17, for use according to embodiment 26, wherein the cognitive dysfunction/impairment is selected from the group comprising: impaired executive functioning, impaired working memory and attention deficits.
E28. The Compound (I) hemiadipate according to any of embodiments 1-17, for use according to any of embodiments 21-22 or 26-27, wherein the patient suffers from schizophrenia.
E29. The Compound (I) hemiadipate according to any of embodiments 1-17, for use according to embodiment 28, wherein the schizophrenic patient is in a clinically stable phase.
E30. The Compound (I) hemiadipate according to any of embodiments 1-17, for use according to any of embodiments 21-22 or 26-27, wherein the patient displays psychosis risk syndrome and/or prodrome of schizophrenia.
E31. The Compound (I) hemiadipate according to any of embodiments 1-17, for use in treating negative and/or cognitive impairments in a patient who is non-schizophrenic.
E32. A method of treatment of persistent negative symptoms, which method comprises the administration of a therapeutically effective amount of Compound (I) hemiadipate according to any of embodiments 1-17 to a patient in need thereof as monotherapy.
E33. A method of treatment of persistent prominent negative symptoms, which method comprises the administration of a therapeutically effective amount of Compound (I) hemiadipate according to any of embodiments 1-17 to a patient in need thereof as monotherapy.
E34. A method of treatment of cognitive impairments, which method comprises the administration of a therapeutically effective amount of Compound (I) hemiadipate according to any of embodiments 1-17 to a patient in need thereof as monotherapy.
E35. Use of Compound (I) hemiadipate according to any of embodiments 1-17 in the manufacture of a medicament for treatment of persistent negative symptoms.
E36. Use of Compound (I) hemiadipate according to any of embodiments 1-17 in the manufacture of a medicament for treatment of persistent prominent negative symptoms.
E37. Use of Compound (I) hemiadipate according to any of embodiments 1-17 in the manufacture of a medicament for treatment of cognitive impairments.
E38. The Compound (I) hemiadipate according to any of embodiments 1-17, for use in a method for treating a patient suffering from persistent negative symptoms, wherein the Compound (I) hemiadipate is administered as monotherapy.
E39. The Compound (I) hemiadipate according to any of embodiments 1-17, for use in a method for treating a patient suffering from persistent prominent or persistent predominant negative symptoms, wherein the Compound (I) hemiadipate is administered as monotherapy.
E40. The Compound (I) hemiadipate according to any of embodiments 1-17, for use in a method for treating a patient suffering from cognitive impairments, wherein the Compound (I) hemiadipate is administered as monotherapy.
E41. A method of treatment of negative symptoms, persistent negative symptoms, prominent negative symptoms, persistent prominent negative symptoms, predominant negative symptoms, persistent predominant negative symptoms and/or cognitive dysfunctions, wherein the patient has clinically stable positive symptoms, said method comprises the administration of a therapeutically effective amount of Compound (I) hemiadipate according to any of embodiments 1-17 as monotherapy.
E42. The pharmaceutical composition according to embodiment 20, characterized in that said composition are manufactured by a process comprising one or more of the process steps selected from wet granulation, fluid bed processing, drying at elevated temperature such as at a temperature above room temperature, aqueous based spray drying, aqueous based coating of granules, pellets or tablets, milling at elevated temperature.
E43. A process for manufacturing the Compound (I) hemiadipate of any one of the embodiments 1-17 comprising the steps:
   a. Obtaining a solution of the base of 5,8-Dimethyl-2-[2-(1-methyl-4-phenyl-1H-imidazol-2-yl)-ethyl]-[1,2,4]triazolo[1,5-a]pyrazine; and
   b. Adding adipic acid to said solution; and
   c. Isolating the resulting Compound (I) hemiadipate by filtrating the solution obtained in step (b).
E44. A process for manufacturing Compound (I) hemiadipate according to embodiment 43, wherein the solution obtained in step (a) is performed in a suitable solvent, such as ethanol.
E45. A process for manufacturing Compound (I) hemiadipate according to either of embodiments 43-44, wherein step (a) and (b) are performed at a suitable temperature, such as about 50-70° C., such as about 60-65° C.
E46. A process for manufacturing Compound (I) hemiadipate according to any of embodiments 43-45, wherein the solution obtained in step (b) is heated to reflux for about 1 hour and cooled to about room temperature before carrying out step (c).

All references, including publications, patent applications and patents, cited herein are hereby incorporated by reference in their entirety and to the same extent as if each reference were individually and specifically indicated to be incorporated by reference and were set forth in its entirety (to the maximum extent permitted by law).

Headings and sub-headings are used herein for convenience only, and should not be construed as limiting the invention in any way.

The description herein of any aspect or aspect of the invention using terms such as “comprising”, “having,” “including” or “containing” with reference to an element or elements is intended to provide support for a similar aspect or aspect of the invention that “consists of”, “consists essentially of” or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

The use of any and all examples, or exemplary language (including “for instance”, “for example”, “e.g.”, “such as” and “as such”) in the present specification is intended merely to better illuminate the invention and does not pose a limitation on the scope of invention unless otherwise indicated.

It should be understood that the various aspects, embodiments, implementations and features of the invention mentioned herein may be claimed separately, or in any combination.

The present invention includes all modifications and equivalents of the subject-matter recited in the claims appended hereto, as permitted by applicable law.

The invention is further described by the following non-limiting Examples.

### EXPERIMENTAL SECTION

### General Materials and Methods:

### Synthesis of Compound (I) Hemiadipate:

The free base of Compound (I) is obtained e.g. via the process described in WO2009/152825 (Example 12). A solvent, such as ethanol, is added to the base of Compound (I) to produce a solution. To the solution of Compound (I) is added adipic acid. All steps described thus far may be carried out at about 60-65° C. The Compound (I) and adipic acid mixture in ethanol is heated to reflux for about 1 hour and cooled to about room temperature over about 2 hours. The Compound (I) hemiadipate is obtained by filtration and may optionally by washed with a solvent, such as ethanol and dried in vacuo at about 50° C.

### Differential Scanning Calorimetry (DSC):

The Differential Scanning Calorimetry (DSC) measurements can be performed using equipment TA-Instruments Discovery DSC. About 2 mg of sample is heated at about 5° C./min under nitrogen flow in a closed pan with a pinhole in the lid.

### Dynamic Vapour Sorption (DVS):

The vapour sorption can be investigated by DVS analysis. The Dynamic Vapour Sorption (DVS) experiments can be performed using a SMS DVS Advantage 01 changing the relative humidity between about 0% to about 95% in steps of about 5-10% RH.

### EXAMPLES

The examples provided below serve to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1: Preparation of Compound (I) Hemiadipate

To the base of Compound (I) (21.8 g) was added ethanol (175 mL); a process for obtaining the free base is described in WO2009/152825 (Example 12). The mixture was heated to 60° C. where Compound (I) completely dissolved. The hot solution was passed through a filter followed by a hot ethanol (22 mL) rinse. The combined filtrates were heated to 60° C. In another flask were added adipic acid (4.82 g) and ethanol (44 mL). The mixture was heated to 65° C. where the resulting solution was passed through a filter. The hot filtrate of adipic acid in ethanol was transferred to the 60° C. hot solution of the base of Compound (I) in ethanol. The flask used to dissolve the adipic acid was rinsed with hot ethanol (22 mL) and passed through the filter and transferred to the hot mixture of compound (I) and adipic acid. The mixture was heated to reflux for 1 hour and then cooled to 20° C. over 2 hours. The hemiadipate of Compound (I) was isolated by filtration and washed two times with ethanol (2×44 mL). The Compound (I) hemiadipate was dried overnight in vacuo at 50° C. Yield: 23.9 g, 90%.

### Example 2: X-Ray Powder Diffractogram (XRPD) Characterization

X-Ray powder diffractograms (XRPD) of Compound (I) hemiadipate were measured on a PANalytical X'Pert PRO X-Ray Diffractometer using CuKα1 radiation (λ=1.5406 Å). The samples were measured in reflection mode in the 2θ-range 3-40° using an X-celerator detector. The XRPD pattern for Compound (I) hemiadipate is shown in FIG. 1. Characteristic main reflections are at the following 2θ-angles: 7.0, 9.9, 10.8, 14.0, 15.3, 16.2, 17.4, 19.1, 19.8 and 20.3. This crystalline form is the only known polymorphic form of Compound (I) hemiadipate.

### Example 3: Differential Scanning Calorimetry (DSC)

The Differential Scanning Calorimetry (DSC) measurements of Compound (I) hemiadipate were performed using equipment TA-Instruments Discovery DSC. About 2 mg of sample was heated at 5° C./min under nitrogen flow in a closed pan with a pinhole in the lid.

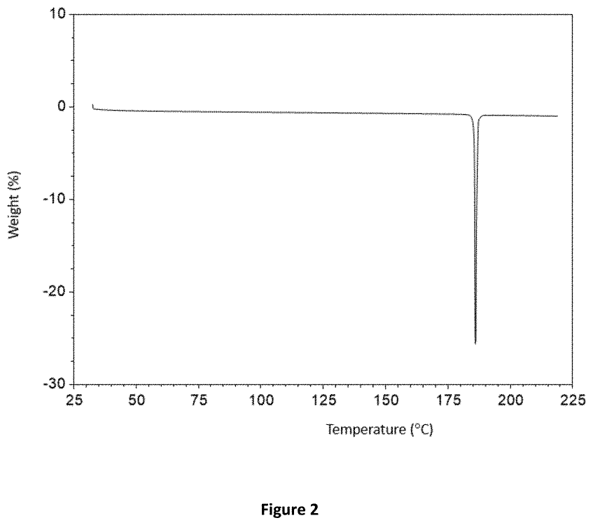

An illustrative DSC result for Compound (I) hemiadipate is shown in FIG. 2. This measurement showed that the compound (I) hemiadipate had an endotherm peak at about 186.0° C. with onset at about 185.6° C.

### Example 4: Dynamic Vapour Sorption (DVS)

The vapour sorption was investigated by DVS analysis. The vapour sorption was found to be less than 0.1% at 95% RH at 25° C.; it can therefore be concluded that the Compound (I) hemiadipate is a non-hygroscopic solid form. On the contrary, the free base was found to be very hygroscopic. The Dynamic Vapour Sorption (DVS) experiments were performed using a SMS DVS Advantage 01 changing the relative humidity between 0% to 95% in steps of 10% RH (in the final step, RH were only increased 5%, from 90 to 95% RH). FIG. 3 shows the resulting DSV curves for Compound (I) hemiadipate (A) and the free base of Compound (I) (B).

The Compound (I) hemiadipate is the only solid form of Compound (I), which is known not to be hygroscopic, see table 2.

### Example 5: Hydrate Formation

To determine whether the Compound (I) hemiadipate are prone to form hydrates it was investigated if precipitation from water equilibrated over 7 days led to a new crystal form. In addition, it was explored if storage of Compound (I) hemiadipate at 95% RH for 5 months led to change in the crystal form. These experiments showed that in both circumstances the obtained solid form of the Compound (I) hemiadipate had not changed from the original form, as confirmed by XRPD. These experiments strongly indicate that Compound (I) hemiadipate does not form hydrates.

The Compound (I) hemiadipate is the only solid form of Compound (I), which is known not to form hydrates, see table 2.

### Example 6: Solvate Formation

To determine whether the Compound (I) hemiadipate forms solvates it was investigated if solvate formation could be observed from precipitation in different organic solvents after equilibration for 4 days in the shake-flask method: Compound (I) hemiadipate was precipitated in various organic solvents from a 1:1 mixture of Compound (I) and adipic acid. The tested solvents were: acetone, acetonitrile, methanol, ethanol, 1-propanol, 2-propanol, 1-butanol, 2-butanol, ethyl acetate, isopropyl acetate, methyl isobutyl ketone, tetrahydrofuran, diethylether, N-methylpyrrolidone, dichloromethane, and n-heptane.

These experiments revealed that for all tested solvents, the XRPD diffractogram of the resulting precipitate were comparable to the XRPD diffractogram shown in FIG. 1 (i.e. the only known crystal form of Compound (I) hemiadipate, which is a non-solvated form). These findings strongly indicate that Compound (I) hemiadipate does not form solvates.

### Example 7: Intrinsic Dissolution Rate (IDR)

Tablets of 10 mg Compound (I) (free base or hemiadipate) were pressed and put into a micro dissolution chamber. It was subsequently measured how fast the substance was dissolved from the well-defined surface of the tablet into the solvent. The length of a full dissolution experiment was 20 min and the IDR was calculated in the range 1-2.5 min, determined as the slope of the dissolution profile in that specific range. The experiment at pH 2 was performed in 0.01 N HCl, at pH 5 in 50 mM acetate buffer and at pH 6.8 in 50 mM phosphate buffer, respectively, all at 37° C. All experiments were performed in duplicates.

Where the tablet was not fully dissolved after end of experiment, the surface was examined by XRPD, to identify if any changes of solid form have occurred during the experiment.

TABLE 1
Summarized IDR data, Compound (I) free
base and Compound (I) Hemiadipate

| | IDR Average | IDR Average |
|---|---|---|
| | mg/cm² × min | mg/cm² × min |
| pH | Compound (I) free base | Compound (I) Hemiadipate |
| 2 | 1.45 | >2* |
| 5 | 0.101 | 0.3 |
| 6.8 | 0.025 | 0.04 |

*Tablet completely dissolved prior to end of experiment (20 min)

These data show that the Compound (I) hemiadipate provides a more desirable dissolution profile for an API, since the dissolution is more rapid compared to the free base of Compound (I).

### Example 8—Efficacy of Compound (I) of the Invention in the Conditioned Avoidance Response (CAR) in Rats in High Dopamine State

In the CAR task, animals are trained to respond to a stimulus within a certain time by moving from one place to another (avoidance), all current antipsychotic agents produce a selective suppression of the avoidance response and the CAR paradigm is therefore considered to possess a high degree of predictive validity for antipsychotic activity (Wadenberg, 2010, Current Pharmaceutical Design, 16(3), 358-370). PDE10A inhibitors are known to reduce this conditioned avoidance response and hence, expected to have effective antipsychotic activity. The inventors of the present invention put forward the hypothesis that many patients with exacerbated positive symptoms can be expected to have increased striatal dopamine levels. Since the dopamine receptors regulate the dopamine signal by controlling cAMP synthesis and PDE10A regulates the gain of this signal by controlling cAMP degradation, the effect of PDE10A inhibition might be altered in conditions with increased striatal dopamine tonus, such as found in patients with prominent positive symptoms.

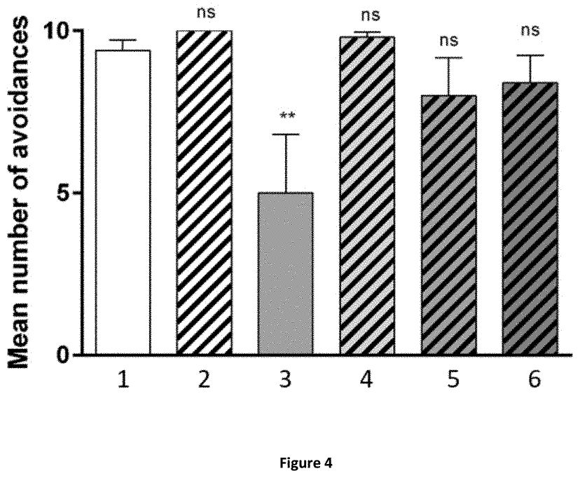

To investigate this further the effect of compounds of the invention were tested in the CAR assay in rats that were co-dosed with a low dose of d-amphetamine (0.20-0.25 mg/kg) to induce a high dopamine state (FIG. 4).

This study confirms that compounds of the invention alone reduced CAR as anticipated. However, it is also shown that combining a low dose d-amphetamine with compounds of the invention (0.3 to 3 mg/kg) (FIG. 1) clearly reduces the capacity of the compounds of the invention to suppress the CAR, even at doses normally leading to full suppression. This suggests that the presumed antipsychotic efficacy of PDE10A inhibitors as measured in the CAR assay are suppressed when the animals are administered a low dose of d-amphetamine to model the higher dopamine levels believed to be associated with strong positive symptoms.

FIG. 4 shows the effect of compounds of the invention in the Conditioned Avoidance Response (CAR) assay in rats as further described in example 8. Compound (I) was tested alone and in combination with a low dose d-amphetamine to induce a high dopamine state. The efficacy was measured as the mean number of avoidances and results are depicted as mean+/−SEM. Compound (I) dosed alone reduced CAR as expected, but when combined with d-amphetamine, this effect was abolished.

The graph displays the effect of Compound (I) alone and in combination with low dose d-amphetamine. Bars represent the following dose groups (left to right): 1) Vehicle; 2) d-amphetamine (0.2 mg/kg); 3) Compound (I) (0.6 mg/kg); 4) Compound (I) (0.3 mg/kg)+d-amphetamine (0.2 mg/kg); 5) Compound (I) (0.6 mg/kg)+d-amphetamine (0.2 mg/kg); and 6) Compound (I) (3.0 mg/kg)+d-amphetamine (0.2 mg/kg). ** p<0.01 compared to vehicle. ns=non-significant.

### Summary of Properties for Solid Forms of Compound (I):

TABLE 2
N.D indicates that the property has never been
determined for that specific solid form

| | Hydrate formation | Hygroscopic |
|---|---|---|
| Solid form of Compound (I) | (Yes/No) | (Yes/No) |
| Free base | Yes | Yes |
| Hemiadipate | No | No |
| Hydrogen Bromide | Yes | Yes |
| Hydrogen Chloride | Yes | Yes |
| Dihydrogen Phosphate | Yes | N.D |
| Hydrogen sulphate | Yes | N.D |
| Citrate | Yes | Yes |
| Fumarate | Yes | N.D |
| Hemi-fumarate | Yes | Yes |
| D-glucoronate | Yes | Yes |
| Glutarate | Yes | N.D |
| 2-oxoglutarate | Yes | Yes |
| L(−)-malate | Yes | Yes |
| Maleate | Yes | Yes |
| Malonate | Yes | Yes |
| Mesylate | Yes | N.D |
| Oxalate | Yes | Yes |
| Hemi-succinate | Yes | Yes |
| L(+)-tartrate | Yes | N.D |
| Tosylate | Yes | Yes |

1. A Compound (I) hemiadipate with the chemical name: 5,8-Dimethyl-2-[2-(1-methyl-4-phenyl-1H-imidazol-2-yl)-ethyl]-[1,2,4]triazolo[1,5-a]pyrazine hemiadipate.

2. The Compound (I) hemiadipate according to claim 1, represented by the formula

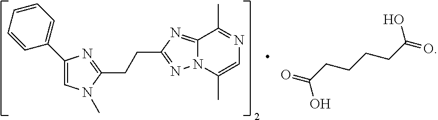

3. The Compound (I) hemiadipate, according to claim 1, wherein said Compound (I) hemiadipate comprises about 2 equivalents of Compound (I) and about 0.8-1.2 equivalent of adipic acid.

4. The Compound (I) hemiadipate according to claim 1, wherein said Compound (I) hemiadipate is crystalline.

5. The Compound (I) hemiadipate according to claim 4, which crystal form is characterized by an XRPD obtained using CuKα1 radiation (λ=1.5406 Å) showing peaks at the following 2θ-angles: 7.0, 9.9, 10.8, 14.0, 15.3, 16.2, 17.4, 19.1, 19.8, 20.3 (±0.1°2θ).

6. The Compound (I) hemiadipate according to claim 4, which crystal form is characterized by an XRPD obtained using CuKα1 radiation (λ=1.5406 Å) showing peaks at the following 2θ-angles: 7.0, 9.9, 10.8, 14.0, 15.3, 16.2, 17.4 (±0.1° 2θ).

7. The Compound (I) hemiadipate according to claim 4, which crystal form is identifiable by an XRPD obtained using CuKα1 radiation (λ=1.5406 Å) as depicted in FIG. 1A.

8. The Compound (I) hemiadipate according to claim 4, which crystal form is characterized by having a DSC trace showing an endotherm with peak at about 185-187° C.

9. The Compound (I) hemiadipate according to claim 1, wherein less than about 1.5% moisture is absorbed when said Compound (I) hemiadipate is exposed to about 95% RH at about 25° C.

10. The Compound (I) hemiadipate according to claim 1, wherein less than about 1.5% moisture is absorbed when said Compound (I) hemiadipate is exposed to about 95% RH at about 25° C., when determined by Dynamic Vapor Sorption (DVS).

11. The Compound (I) hemiadipate according to claim 1, wherein less than about 1.5% moisture, preferably less than 1.0%, 0.5% or 0.3% or most preferably less than 0.1% is absorbed when said Compound (I) hemiadipate is exposed to about 95% RH at about 25° C., when determined by Dynamic Vapor Sorption (DVS) according to the method of Example 4.

12. (canceled)

13. (canceled)

14. A pharmaceutical composition comprising the Compound (I) hemiadipate according to claim 1 and at least one pharmaceutically acceptable excipient.

15. The pharmaceutical composition according to claim 14, characterized in that said composition is manufactured by a process comprising one or more of the process steps selected from wet granulation, fluid bed processing, drying at elevated temperature such as at a temperature above room temperature, aqueous based spray drying, aqueous based coating of granules, pellets or tablets, and milling at elevated temperature.

16. A process for manufacturing the Compound (I) hemiadipate of claim 1 comprising the steps:
a. Obtaining a solution of the base of 5,8-Dimethyl-2-[2-(1-methyl-4-phenyl-1H-imidazol-2-yl)-ethyl]-[1,2,4]triazolo[1,5-a]pyrazine;
b. Adding adipic acid to said solution; and
c. Isolating the resulting Compound (I) hemiadipate by filtrating the solution obtained in step (b).

17. The process for manufacturing Compound (I) hemiadipate according to claim 16, wherein the solution obtained in step (a) comprises ethanol.

18. The process for manufacturing Compound (I) hemiadipate according to claim 16, wherein step (a) and (b) are performed at a temperature of about 50-70° C.

19. The process for manufacturing Compound (I) hemiadipate according to claim 16, wherein the solution obtained in step (b) is heated to reflux for about 1 hour and cooled to about room temperature before carrying out step (c).

20. A method of treating persistent prominent negative symptoms associated with schizophrenia, persistent prominent negative symptoms associated with schizophrenia, or cognitive impairments associated with schizophrenia in a subject in need thereof, comprising administering to the subject Compound (I) hemiadipate according to claim 1.

21. A method of treating negative and/or cognitive impairments in a subject who is non-schizophrenic, comprising administering to the subject Compound (I) hemiadipate according to claim 1.
